(19) United States
(12) Patent Application Publication
(10) Pub. No.: US 2017/0000945 A1
(43) Pub. Date: Jan. 5, 2017

(54) METHOD AND DEVICE FOR CONTROLLING THE FLOW THROUGH A MEDICAL INFUSION LINE

(71) Applicant: B. BRAUN MELSUNGEN AG, Melsungen (DE)

(72) Inventor: KARSTEN HASLBECK, MELSUNGEN (DE)

(73) Assignee: B. BRAUN MELSUNGEN AG, Melsungen (DE)

(21) Appl. No.: 15/113,613

(22) PCT Filed: Jan. 19, 2015
(86) PCT No.: PCT/EP2015/050899

§ 371 Date: Jul. 22, 2016

(30) Foreign Application Priority Data
Jan. 23, 2014 (DE) 10 2014 201 258.9

(51) Int. Cl. A61M 5/168

(52) U.S. Cl. CPC A61M 5/16854; A61M 2205/3306; A61M 2205/3348; A61M 2205/3355

(57) ABSTRACT

An improved device for simple and reliable control of flow through a medical infusion line, one end of the medical infusion line having a fluid conveying pump provided on it and the other end assigned to a patient, the infusion line forming a main flow path from the fluid conveying pump to the patient-side end. Fluid is conveyed along the main flow path through the infusion line by means of the fluid conveying pump. At least a part of the fluid is introduced, along a measurement flow path branching off from the main flow path at a branching point, from the infusion line into a measurement reservoir connected to the infusion line. The infusion line includes a fluid restrictor between the fluid conveying pump and the branching point, and the main flow path as viewed in direction of the flow is interrupted after the branching point of the measurement flow path for filling the measurement reservoir by the pressure of the fluid conveying pump. The fluid in the measurement reservoir is detected.

### CROSS REFERENCE TO RELATED APPLICATIONS

This application is the U.S. national phase application of PCT International Application No. PCT/EP2015/050899 filed Jan. 19, 2015, which claims priority to German Patent Application No. DE 10 2014 201 258.9 filed Jan. 23, 2014, the contents of each application being incorporated by reference herein.

### FIELD OF THE INVENTION

The invention relates to a method and a device for controlling the flow through a medical infusion line, wherein one end of the medical infusion line has a fluid conveying pump provided on it and the other end thereof is assigned to a patient, the infusion line forming a main flow path from said fluid conveying pump to the patient-side end.

### BACKGROUND OF THE INVENTION

Such infusion lines are used for continuous administration of a medicament in fluid form to a patient. For instance, medicaments are administered continuously in small doses. In such cases, the fluid will be conveyed in extremely low flow rates in the range from 0.5 to 500 ml per hour. If the pump should happen to fail or the conveyance of fluid should be impeded for some other reason, this can be detected only at an advanced point in the infusion period. Particularly in case of low flow rates, a weight or volume reduction of the liquid reservoir of the pump has been barely visible and has not been measurable while keeping the resultant expenditure on a practicable level. Thus, the need exists to be able to detect in a fast and simple manner whether fluid is being conveyed through the infusion line.

Measuring methods known as of yet are based on the principal concept of measuring the throughflow rate within the flow. In view of the low flow rates existing in infusion therapy or in analgesics infusion, those mechanical approaches wherein the flow rate is determined with the aid of component parts being mechanically moved by the flow, are not eligible. Such flow rates are not sufficient for causing a movement of mechanical parts. Instead, the mechanical parts will be bypassed by the fluid flow without effecting a pulse transmission that would generate a mechanical movement. As an alternative, electronic approaches are used wherein the throughflow of the fluid is detected with the aid of electronic sensors. These electronic approaches on the one hand are expensive and, on the other hand, will require an external energy source. An elastomeric infusion pump, however, should be operable independently of external energy sources.

### SUMMARY OF THE INVENTION

It is an object of the invention to render possible a technically simple and reliable control of the throughflow in a medical infusion line.

The device according to aspects of the invention is defined by each of the independent claims.

Through the infusion catheter, the main flow path leads from the fluid conveying pump to the patient-side end. Arranged on the patient-side end is a connector serving for connection with the patient or with components inserted into the patient. Thus, under the technical aspect, the patent end is to be considered as a connection site of the patient. While the previously known throughflow control methods involved the necessity to perform the flow detection at a site within the flow passing through the main flow path, the invention relates to the idea to redirect the fluid flow into a separate measurement reservoir, wherein the main flow path is provided with a flow restrictor arranged before or after the measurement flow path when viewed in the direction of the patient-side end. In this situation, the fluid conveying pump is continued to be operated in an unchanged manner, and the entire energy of the fluid flow can be used for control of the throughflow. First, in the process, the fluid which during operation of the pump is flowing from the main flow path into the measurement reservoir can be considered as an indicator confirming the general operativeness of the pump.

For control of the throughflow, the main flow path will be interrupted, wherein two variants can be envisioned:

As a first variant, the main flow path can be interrupted between the fluid conveying pump and the branching point of the measurement flow path from the main flow path. Thus, in this case, the main flow path will be interrupted in flow direction before the measurement reservoir, and the fluid conveying pump will not convey further fluid into the measurement reservoir and toward the patient-side end. After such an interruption of the main flow path, the fluid in the measurement reservoir will be monitored. If fluid does flow out, the infusion line is open to flow, and the throughflow to the patient is guaranteed. If no fluid flows out from the measurement reservoir, this is an indicator signaling that the infusion line in the direction of the patient-side end and/or the throughflow to the patient is interrupted or impaired. In this first variant, a fluid restrictor is provided in the infusion line along the main flow path between the branching point of the measurement flow path and the patient-side end. This variant is known from the state of the art and does not form a part of the invention.

In a variant according to aspects of the invention, the main flow path will be interrupted between the branching point of the measurement flow path and the patient-side end, i.e. behind the branching point of the measurement flow path and the measurement reservoir as viewed in the flow direction. In this variant, a flow restrictor is arranged in the infusion line along the main flow path between the fluid conveying pump and the branching point of the measurement flow path. Thus, the flow restrictor is here arranged, as viewed in flow direction, before the branching point of the measurement flow path and the measurement reservoir. Upon interruption of the main flow path behind the branching point of the measurement flow path as viewed in flow direction, the fluid conveyed by the infusion line will flow completely into the measurement reservoir. In this situation, the quantity of the inflowing fluid will be monitored and serves as an indicator of the operativeness of the fluid conveying pump or the openness to flow of the infusion line and respectively of the main flow path between the pump and the measurement reservoir.

A flow restrictor as mentioned in the present context is generally to be understood as a flow resistor for reducing the flow. The flow resistor can be realized as a separate component or by a suitable cross section of the infusion line.

After the throughflow and the operativeness of the infusion line assembly have been verified with the fluid in the measurement reservoir, the fluid path to the patient will be opened again. Thereupon, the fluid collected in the measurement reservoir will flow out of the measurement reservoir and will be supplied to the patient via the main flow path. Thus, the throughflow measurement will not cause a loss of fluid.

In the process, the fluid received in the separate measurement reservoir can be used for wetting a prism so as to change the light refraction of the prism. For instance, a colored layer can be provided under the prism, which layer will be visible only if the surface of the prism has been wetted with fluid, whereas, in a dry environment, the path of the light rays within the prism will prevent the visibility of said colored layer.

A further principle for detection of fluid within the measurement reservoir can consist in providing a piston in the measurement reservoir which, under the effect of the inflowing fluid, will—against the force of a spring—be displaced in a manner visible from the outside. In this case, the fluid pumped into the measurement reservoir by the fluid conveying pump will displace the piston. The displacement or deflection of the piston can be rendered visible in different manners. For instance, the piston itself can be visible or be designed to shift an indicator element along a scale.

A further alternative for detection of fluid within the measurement reservoir can reside in a visible change of shape of the measurement reservoir in dependence on the quantity of liquid in the reservoir. For instance, the measurement reservoir can be realized as a balloon adapted to expand depending on the quantity of liquid. There can also be conceived a manometer, a (miniature) bellows or an elastic tube which in the empty state is curved and in the filled state is stretched. Further, for each type of measurement reservoir, it shall be envisioned to provide a pointer which, via a leverage effect, will enhance the display.

Particularly, the inventive device for flow control can be a part of a PCA (Patient Controlled Analgesia) device and/or be used in connection with a flow selector.

The fluid pump can be an elastomeric pump, a spring pump, a vacuum pump or a syringe pump.

### BRIEF DESCRIPTION OF THE DRAWINGS

The invention is best understood from the following detailed description when read in connection with the accompanying drawings. Included in the drawings are the following figures:

FIG. 1 is a view of an embodiment in a first operating state in accordance with the prior art,

FIG. 2 is a view of the embodiment of FIG. 1 in a second operating state in accordance with the prior art,

FIG. 3 is a view of an exemplary embodiment of the invention in a first operating state,

FIG. 4 is a view of the exemplary embodiment of FIG. 3 in a second operating state,

FIG. 5 is a view of a further exemplary embodiment which is not part of the invention, in a first operating state in accordance with the prior art,

FIG. 6 is a view of the embodiment of FIG. 5 in a second operating state in accordance with the prior art,

FIG. 7 is a view of another exemplary embodiment of the invention in a first operating state,

FIG. 8 is a view of the exemplary embodiment of FIG. 7 in a second operating state,

FIG. 9 is a view of a detail of a further exemplary embodiment,

FIG. 10 is a view of the exemplary embodiment according to FIG. 9 in a different operating state,

FIG. 11 is a view of a detail of a further exemplary embodiment,

FIG. 12 is a view of a detail of a further exemplary embodiment,

FIG. 13 is a view of a detail of a further exemplary embodiment,

FIG. 14 is a view of a detail of a further exemplary embodiment,

FIG. 15 is a view of the exemplary embodiment according to FIG. 14 in a different operating state,

FIG. 16 is a view of a detail of a further exemplary embodiment,

FIG. 17 is a view of the exemplary embodiment according to FIG. 16 in a different operating state,

FIG. 18 is a view of a detail of a further exemplary embodiment, and

FIG. 19 is a view of the exemplary embodiment according to FIG. 18 in a different operating state.

### DETAILED DESCRIPTION OF THE PREFERRED EMBODIMENTS

All exemplary embodiments are related to the principle of an infusion line assembly consisting of an infusion line 12, a fluid conveying pump 14 and a device 16 for controlling the throughflow through said infusion line 12. The infusion line 12 has two ends 18, 20, the first end 18 among them being connected to said fluid conveying pump 14 and the second end 20 being assigned to a patient. The second end 20 is assigned to a patient in the sense that it comprises a connector 22 which is connectible to the patient or to a catheter inserted into the patient.

Infusion line 12 comprises a main flow path 24 extending through the infusion line from said one end 18 thereof to said other end 20 so that fluid can be conveyed along said path from pump 14 to connector 22.

Infusion line 12 is provided with a branching point 26 connected to a measurement reservoir 28. Said branching point 26 and said measurement reservoir 28 form said device 16 for throughflow control. In this arrangement, the branching point 26 can be realized as an integral part of infusion line 12 or be provided as a component connectible to infusion line 12 at a later time.

Branching point 26 forms a measurement flow path 30 branching off from said main flow path 24 and entering into the measurement reservoir 28.

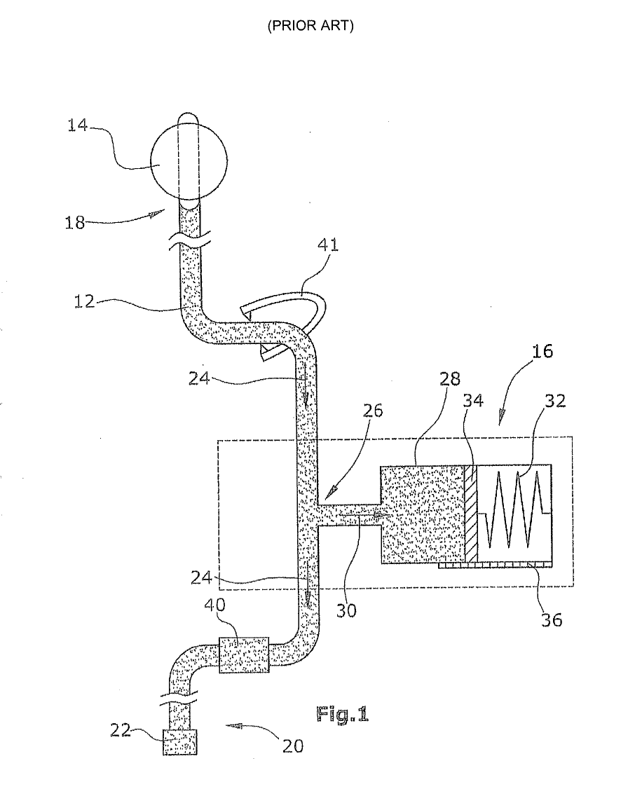

In the embodiment according to FIG. 1, the measurement reservoir 28 includes, in its interior, a piston 34 which is displaceable against the force of a spring 32 in such a manner that the fluid flowing along said measurement flow path 30 will displace the piston 34 within the measurement reservoir 28 against the spring force. The displacement of piston 34 is indicated on a scale 36. Between the fluid pump 14 and branching point 26, infusion line 12 can be clamped shut with a clamp 41 so as to interrupt the main flow path 24. Between branching point 26 and patient connector 22, infusion line 12 comprises a flow restrictor 40.

In the operating state according to FIG. 1, the clamp 41 is opened and the main flow path 24 is not interrupted. The fluid pump 14 will convey the fluid, represented by dots, along the main flow path 24 via infusion line 12 to the patient-side end 20 of the latter and via branching point 26 along the measurement flow path 30 into the measurement reservoir 28. In the process, the fluid pump 14 will build up, in measurement reservoir 28, a pressure acting on piston 34, which pressure will act against the spring force of spring 32 and will displace the piston 34. The displacement of piston 34 as visible on scale 36 can serve as an indicator of the operation or the functional operability of the fluid pump 14.

For examining whether the infusion line 12, the fluid restrictor 40, the patient connector 22 and possible additional components farther downstream, such as e.g. filters, catheters etc. are unobstructed and functional, said clamp 41 will be briefly closed. For this purpose, clamp 41 should not be a locking clamp but should automatically open when released. Alternatively, the infusion line 12 can also be briefly pressed together or kinked by hand for interrupting the fluid flow. During the clamped state of infusion line 12, operation of fluid pump 14 will be continued. However, no further fluid will be conveyed into the measurement reservoir 28, and the fluid pressure generated by fluid pump 14 will not act on the piston 34 anymore. The spring force will displace the piston, and the fluid will be conveyed from the measurement reservoir 28 and into the infusion line 12 in the direction of patient-side end 20 when the infusion line 12 and all following components are open to flow. The term “open to flow” is meant in the sense that the fluid is being conveyed and that the fluid flow is not blocked or reduced by damage, kinking or obstruction. In this regard, the displacement of piston 34 serves as a measure of the openness to flow of infusion line 12 along main flow path 24 in the direction of the patient. If the infusion line 12 or one of the components connected to it is damaged and blocks the fluid flow, the piston 34 will press out less or no fluid from measurement reservoir 28. The displacement of piston 34 will then be different from the one in case of an infusion line 12 that is open to flow.

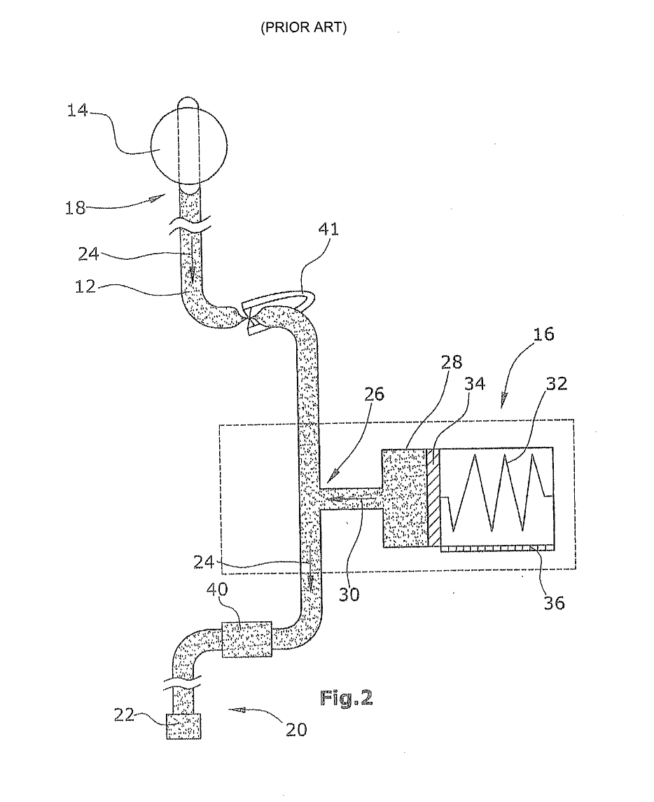

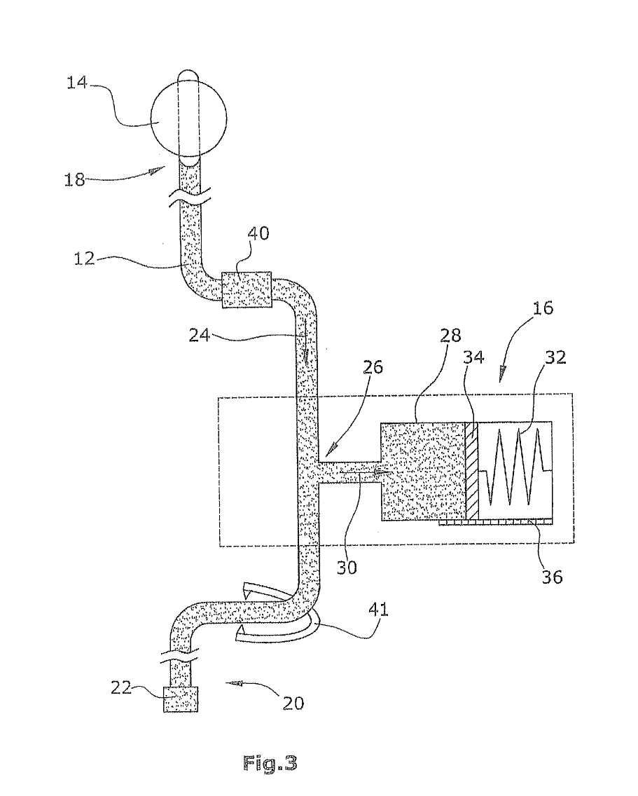

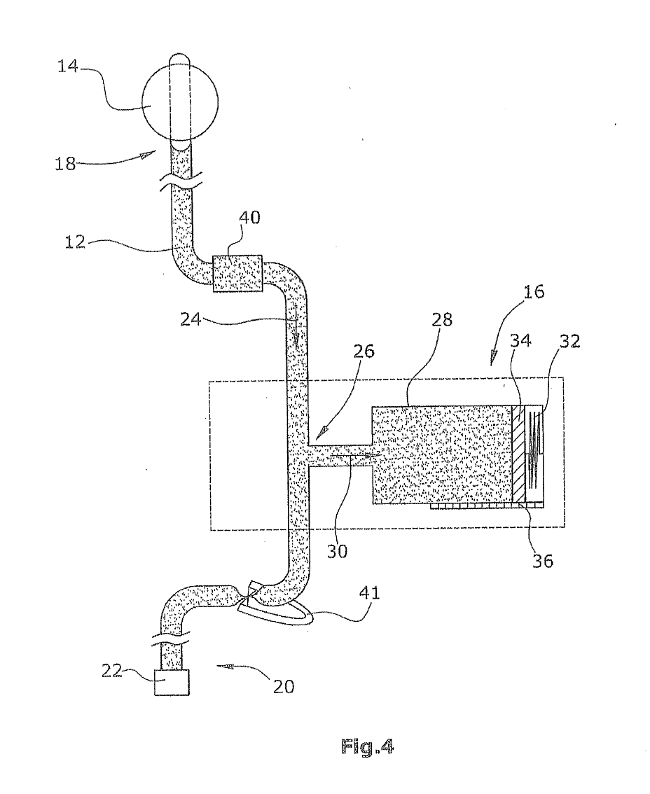

The embodiment according to FIGS. 3 and 4 is different from the embodiment according to FIGS. 1 and 2 only by the arrangement of clamp 41 and flow restrictor 40. In the second exemplary embodiment, flow restrictor 40 is arranged between fluid pump 14 and branching point 26. Clamp 41 serves for interrupting the main flow path 24 in the area between branching point 26 and patient end 20. In the non-clamped state according to FIG. 3, the operating state will then correspond to that according to FIG. 1. The clamp serves, and can be considered as, a control unit on the one hand and as a simulation of a blockade on the other hand.

The second operating state according to FIG. 4, however, is different from the second operating state of the first embodiment according to FIG. 2. In FIG. 4, when infusion line 12 is in its clamped-shut condition, fluid pump 14 will continue to be operated, and fluid will continue to be conveyed into measurement reservoir 28. Since no fluid can flow anymore in the direction of patient-side end 20, fluid pump 14 will build up an ever more increasing fluid pressure within measurement reservoir 28. The resulting displacement of piston 34 will then serve as an indicator of the operability of fluid pump 14 and the openness to flow of infusion line 12 in the area between fluid pump 14 and measurement reservoir 28. Also in FIG. 4, clamp 41 (as a control unit) will be closed only briefly so that the interruption of the infusion will be short and the overall quantity of the fluid administered to the patient will not decrease. In case of a blockade (on the patient), this unit will function “automatically” (the liquid column would rise).

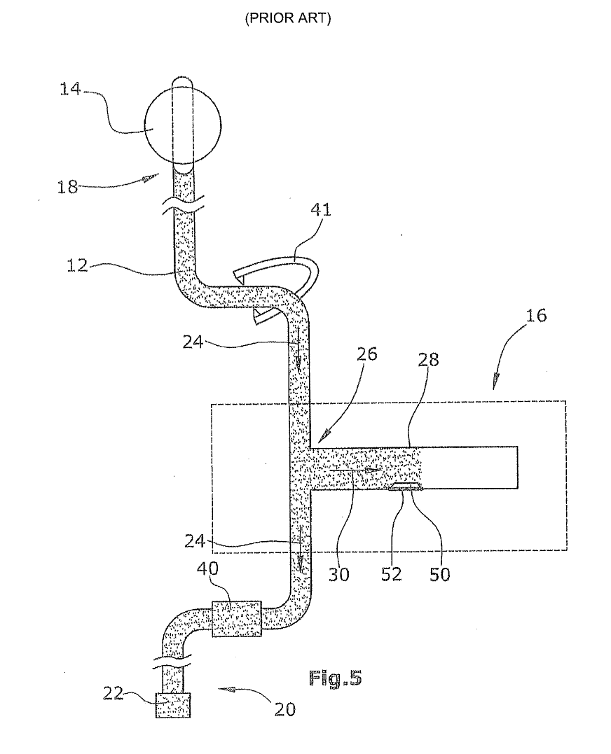

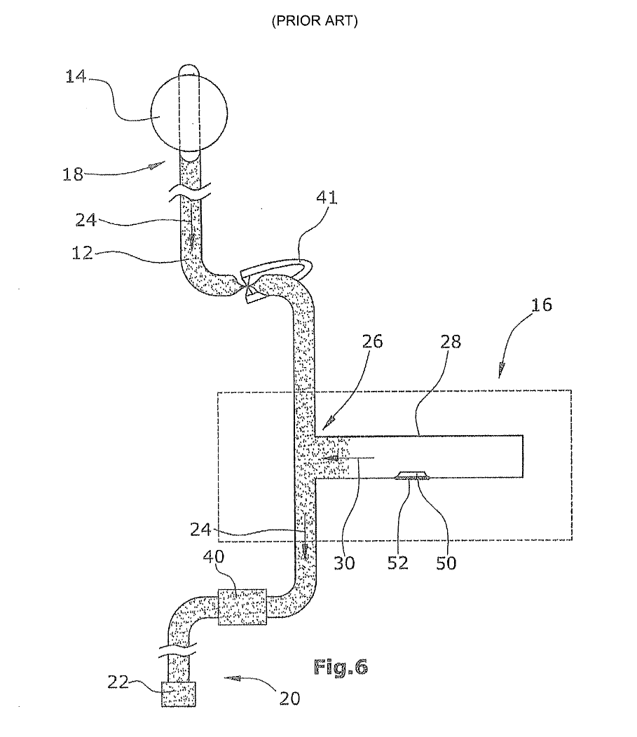

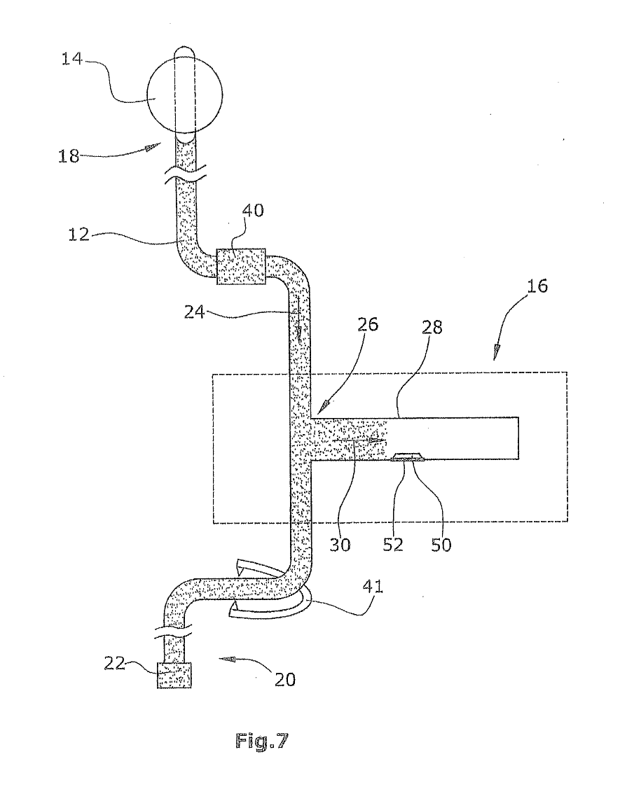

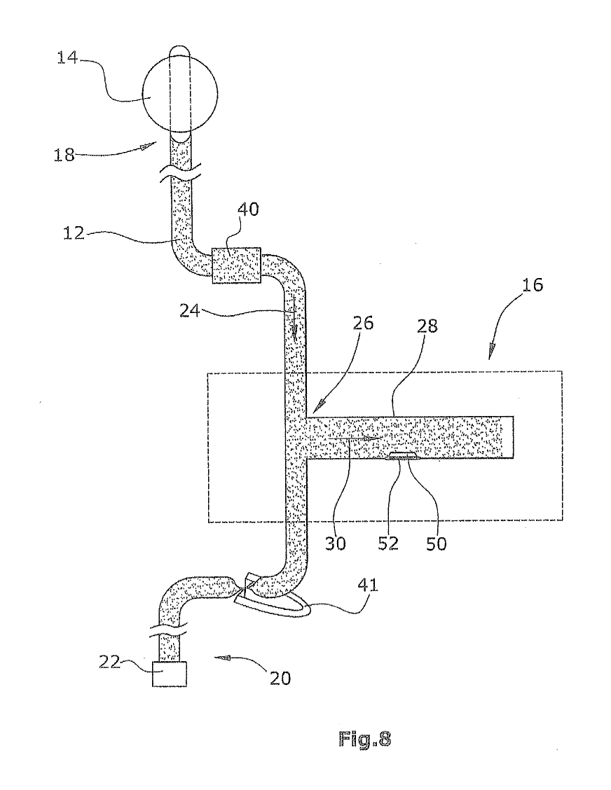

The third embodiment according to FIGS. 5 and 6 corresponds to the first exemplary embodiment according to FIGS. 1 and 2 except for the device 16 for control of the throughflow. Correspondingly, the fourth exemplary embodiment according to FIGS. 7 and 8 is different from the second exemplary embodiment according to FIGS. 3 and 4 only by the device 16. In the exemplary embodiment according to FIGS. 3 and 4, the devices 16 for controlling the throughflow are identical. The difference from the exemplary embodiment according to FIGS. 1 and 2 resides in that the measurement reservoir does not comprise a piston 34 displaceable against the force of a spring 32 but instead comprises a prism 50 on whose bottom a colored layer 52 e.g. in red color is provided. Said prism is light-transmissive and is designed to the effect that, in the state illustrated in FIG. 6, it will reflect light completely when in a dry environment so that the colored layer 52 will not be visible. In the states shown in FIGS. 5 and 8, the prism 52 is wetted by the fluid while no total reflection will occur anymore and the colored layer 52 will be visible. This has the consequence that, in case of a functioning, sufficient throughflow, the device 16—due to the special refraction conditions of prism 50—will present the colored layer 52 as an indicator confirming a correct throughflow. If, however, the measurement reservoir 28, as e.g. in FIG. 6 or in case of a defect fluid conveying pump, does not contain fluid and the prism 50 is surrounded by a dry environment, the colored layer 52 will not be presented.

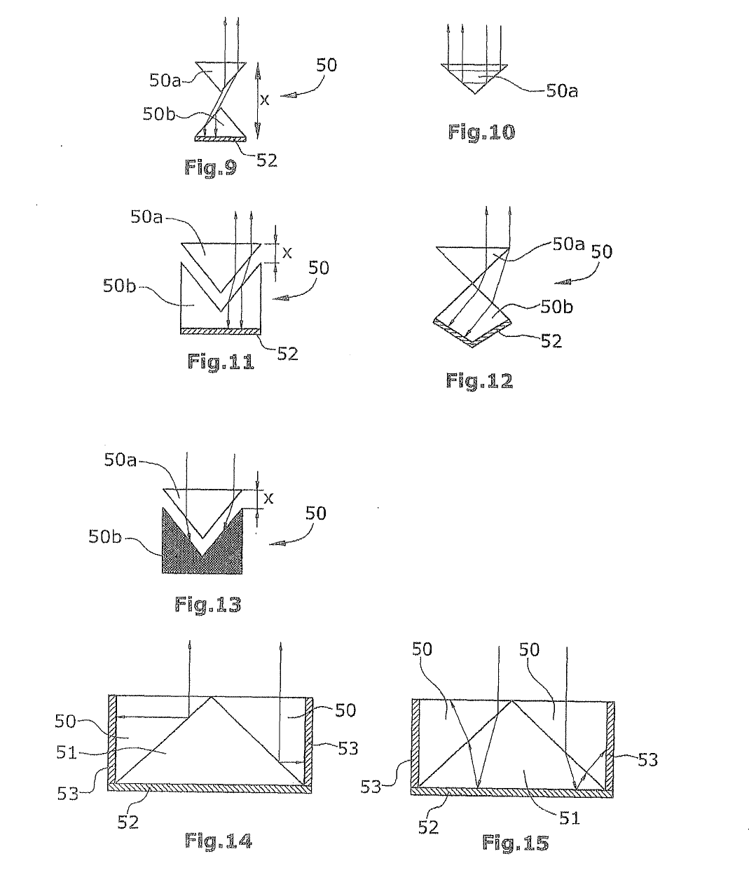

FIGS. 9-15 illustrate various exemplary embodiments of such a prism 50. FIGS. 9-13 herein show two-part prisms 50 comprising an upper part 50a and a lower part 50b. In FIGS. 9, 11 and 12 the bottom of the lower part 50b of the prism is provided with a colored layer 52. In FIG. 13, there does not exist a separate colored layer but, instead, the lower part 50b of the prism is colored. FIGS. 9, 11, 12 and 13 illustrate the path of rays of the light through the prism 50 when the prism has been wetted with fluid, i.e. in the operating states shown to FIGS. 5 to 8. FIG. 10 illustrates the upper part 50a of the prism according to the exemplary embodiments shown in FIG. 9 in a dry environment in which the light is reflected totally and the colored layer 52 is not visible. This is the case in the operating state according to FIG. 6.

FIGS. 14 and 15 show an exemplary embodiment of a two-part prism 50 whose two parts together with the colored bottom 52 together enclose a throughflow channel 51 for the fluid. Herein, the prism is arranged in the measurement reservoir 28 in a manner causing the fluid contained in measurement reservoir 28 to flow into the channel 51. FIG. 14 shows the path of rays in the operating state according to FIGS. 6 and 7, i.e. in a dry environment. In this situation, prism 50 will reflect the incident light onto a lateral colored layer 53 e.g. in red color. Thus, in a dry environment according to FIG. 6, the red color is visible. FIG. 15 shows the path of rays in the operating states according to FIGS. 5 and 8 in which the channel 51 has fluid streaming through it. This will result in the path of rays shown in FIG. 15 wherein light is reflected on the colored bottom 52. In this situation, the colored layer 52 is kept in green color so that the prism in FIG. 15 will present the green color.

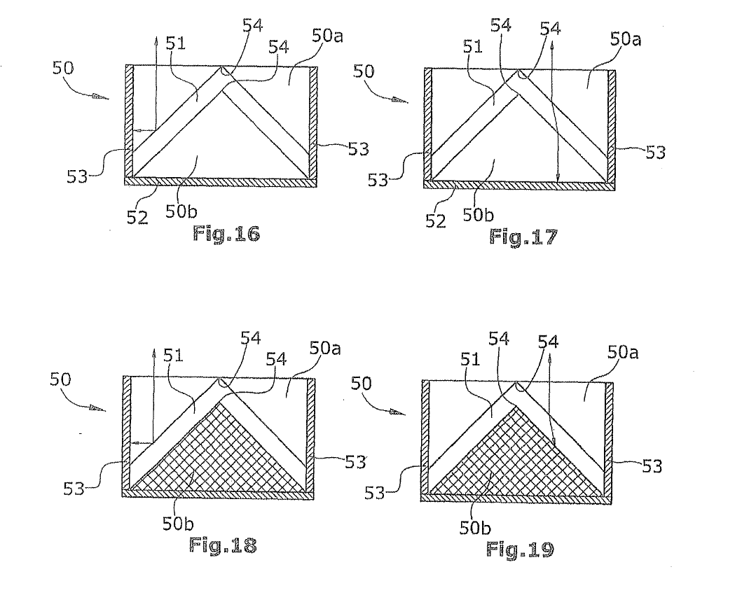

FIGS. 16 and 17 show an exemplary embodiment of a two-part prism 50 comprising two part-prisms 50a and 50b. All part-prisms 50a, 50b are rectangular, i.e. they are provided with a rectangular tip 54. Between the two prisms 50a, 50b, a channel 51 is provided for throughflow of the fluid. At the lateral edges, a colored layer 53 in a first color (e.g. red) is provided which, in the dry state shown in FIG. 16, will reflect the light. Thus, in a dry environment, the red colored layer is visible. FIG. 17 shows the path of rays when fluid is present in channel 51. In this case, the incident light is reflected onto a lower colored layer 52 having a second color differing from the first color (green). Thus, when fluid is present in channel 51, the green colored layer is visible.

The exemplary embodiment according to FIGS. 18 and 19 is different from the exemplary embodiment according to FIGS. 16 and 17 only in that no lower colored layer 52 is provided under the second part-prism 50b but, instead, the second prism 50b is colored in said second color differing from the first color (green). In the dry state without fluid in channel 51 as depicted in FIG. 18, the light will be reflected by the red colored layer 53 as shown in FIG. 16. In the state shown in FIG. 19, with channel 51 having a flow passing through it, the light will be reflected by the green part-prism 50, and the green coloring of prism 50b will be visible.

1-11. (canceled)

12. A device for controlling flow through a medical infusion line, one end of said medical infusion line having a fluid conveying pump provided on it and the other end thereof being assigned to a patient, the infusion line forming a main flow path from the fluid conveying pump to the patient-side end, wherein:
a) fluid is conveyed along the main flow path through the infusion line the fluid conveying pump;
b) at least a part of the fluid is introduced, along a measurement flow path branching off from the main flow path at a branching point, from the infusion line into a measurement reservoir connected to the infusion line;
c) the infusion line comprises a fluid restrictor between the fluid conveying pump and the branching point, and the main flow path as viewed in direction of the flow is interrupted after the branching point of the measurement flow path for filling the measurement reservoir by the pressure of the fluid conveying pump;
d) the fluid in the measurement reservoir is detected.

13. The device of claim 12, wherein the fluid detection in the measurement reservoir is performed on the basis of a visible displacement of a piston which, against the force of a spring, is displaced by fluid flowing into the measurement reservoir.

14. The device of claim 12, wherein the fluid detection in the measurement reservoir is performed on the basis of a visible change of shape of the measurement reservoir under the effect of the pressure of the fluid flowing into the measurement reservoir.

15. The device of claim 12, wherein the fluid detection in the measurement reservoir is performed by monitoring a prism, particularly a rectangular prism, accommodated in the measurement reservoir and wettable by the fluid, said prism, when wetted by fluid, refracting light in a different manner than in a non-wetted state.

16. The device of claim 12, wherein the main flow path is interrupted, as viewed in flow direction, before the branching point of the measurement flow path for evacuating the previously filled measurement reservoir toward the patient-side end.

17. The device of claim 12, wherein the interruption of the main flow path is automatically discontinued behind the branching point as soon as a predefined quantity of fluid has flown into the measurement reservoir or has flown out from it.

18. The device of claim 12, wherein the interruption of the flow path is performed during the fluid measurement by active application of an external force, wherein, upon removal of said force, the interruption is then automatically discontinued.

19. A device for control of flow through a medical infusion line, one end of said medical infusion line having a fluid conveying pump provided on it and the other end thereof being assigned to a patient, the infusion line forming a main flow path from the fluid conveying pump to the patient-side end, said device comprising a measurement reservoir connectible to the infusion line via a measurement flow path, said measurement flow path branching off from the main flow path at a branching point and the measurement reservoir being not contained in the main flow path, and the infusion line comprising a fluid restrictor between the fluid conveying pump and the branching point.

20. The device according to claim 19, wherein the measurement reservoir comprises a spring and a piston displaceable against the force of the spring by fluid flowing into the measurement reservoir.

21. The device of claim 19, wherein, in the measurement reservoir, a prism is provided, said prism being wettable by fluid flowing into the measurement reservoir and, when wetted by fluid, refracting light in a different manner than in a dry environment.

22. An infusion line assembly comprising a fluid conveying pump, an infusion line, one end of said infusion line being provided with the fluid conveying pump and the other end thereof being assigned to a patient, said infusion line forming a main flow path from the fluid conveying pump to the patient-side end, and comprising the control device of claim 19.
